(19) United States
(12) Patent Application Publication
(10) Pub. No.: US 2015/0148601 A1
(43) Pub. Date: May 28, 2015

(54) CATHETERS WITH INDENTED MARKERS

(71) Applicant: Boston Scientific Scimed, Inc., Maple Grove, MN (US)

(72) Inventors: Jason Weiner, Grafton, MA (US); Anne Sluti, Brighton, MA (US)

(21) Appl. No.: 14/549,308

(22) Filed: Nov. 20, 2014

Related U.S. Application Data
(60) Provisional application No. 61/908,836, filed on Nov. 26, 2013.

(51) Int. Cl. A61M 25/00; A61B 1/06; A61B 1/012; A61B 1/05

(52) U.S. Cl. CPC A61M 25/0068; A61B 1/05; A61B 1/06; A61B 1/012; A61M 2025/0008
USPC 600/109; 604/523

(57) ABSTRACT

A catheter may include an elongate tubular member having an outer wall and an inner wall defining a wall thickness. The inner wall may define a lumen extending therethrough. The elongate tubular member may include a first plurality of recessed portions disposed in a distal section, wherein the first plurality of recessed portions each includes a visual marker disposed therein.

### REFERENCE TO RELATED APPLICATIONS

This application claims priority under 35 U.S.C. §119(e) to U.S. Provisional Application No. 61/908,836, filed Nov. 26, 2013, the entire disclosure of which is herein incorporated by reference.

### FIELD

Embodiments of the disclosure are generally directed to systems and devices including indentations with markings for locating catheters during procedures.

### BACKGROUND

During some endoscopic procedures, a medical device such as a catheter may extend and retract in and out of an endoscope many times. Some medical devices used through the endoscope may require visual markers to serve as positioning guides or warning indicators (e.g. BT catheter, biopsy, forceps, tomes, etc.). The visual markers may be coated on an outer surface of the medical devices. In some instances, some visual markers are applied in the form of bands over the medical devices. However, the visual markers may rub off as the medical device extends or retracts from the endoscope and therefore, no longer function as positioning guides or warning indicators.

Thus, there may exist a need for systems and devices having visual markers applied in a way that the visual markers may be maintained during extension or refraction from the endoscope.

### SUMMARY

A catheter may include an elongate tubular member having an outer wall and an inner wall defining a wall thickness. The inner wall can further define a lumen extending therethrough. The elongate tubular member may include a first plurality of recessed portions disposed in a distal section. Each of the first plurality of recessed portions may include a visual marker disposed therein.

An endoscopic system may include an endoscope having a lumen extending therethrough. The endoscope may include a light source and a camera objective each disposed at a distal end for observation distal of the distal end. The endoscopic system may also include an elongate tubular member having a first plurality of recessed portions disposed in a distal section thereof. Each of the first plurality of recessed portions may include a first visual marker disposed therein. The endoscope may be configured to slidably receive the elongate tubular member within the lumen.

### BRIEF DESCRIPTION OF THE DRAWINGS

FIG. 1 illustrates an example endoscopic system;

FIG. 2 illustrates an example catheter for use with the endoscopic system of FIG. 1;

FIG. 3 illustrates an enlarged portion of the example catheter of FIG. 2;

FIG. 4 is a partial side view of an example catheter in accordance with the present disclosure;

FIG. 5 is a partial cross-sectional view of the example catheter of FIG. 4; and

FIG. 6 illustrates an example endoscopic system, including the example catheter of FIG. 4.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the present disclosure.

### DETAILED DESCRIPTION

For the following defined terms, these definitions shall be applied, unless a different definition is given in the claims or elsewhere in this specification.

The terms “proximal” and “distal” shall generally refer to the relative position, orientation, or direction of an element or action, from the perspective of a clinician using the medical device, relative to one another. While the terms are not meant to be limiting, “proximal” may generally be considered closer to the clinician or an exterior of a patient, and “distal” may generally be considered to be farther away from the clinician, along the length of the medical device.

All numeric values are herein assumed to be modified by the term “about,” whether or not explicitly indicated. The term “about”, in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified.

Weight percent, percent by weight, wt %, wt-%, % by weight, and the like are synonyms that refer to the concentration of a substance as the weight of that substance divided by the weight of the composition and multiplied by 100.

The recitation of numerical ranges by endpoints includes all numbers within that range, including the endpoints (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following description should be read with reference to the drawings wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The various individual elements described below, even if not explicitly shown in a particular combination, are nevertheless contemplated as being combinable or arrangable with each other to form other additional embodiments or to complement and/or enrich the described embodiment(s), as would be understood by one of ordinary skill in the art.

During endoscopic procedures, some exemplary medical devices such as a catheter, a balloon catheter, an ablation catheter, etc. may extend and retract in and out of an endoscope many times to perform medical procedures. For such procedures, the endoscope is first inserted into a patient's body. An endoscope may include a light source and a camera objective or other viewing means disposed at a distal end thereof for observation distal of the distal end. For example, the endoscope may be configured to view internal organs of a patient's body. Thereafter, a medical device, a catheter for example, is inserted into the patient's body by passing it through the endoscope. The endoscope may traverse through circuitous vessels or passageways to reach a target site. In order to track location or position of the catheter within the patient's body and/or relative to the endoscope, visual markers may be disposed on the catheter. Some visual markers may be radiopaque, which can be imaged using fluoroscopy or X-ray imaging. When the catheter extends and retracts in and out of the endoscope, an outer wall of the catheter may frictionally contact internal walls of the endoscope. In addition or alternatively to being extended and retracted in and out of an endoscope, a catheter or guidewire having visual markers may be extended in and out of (i.e., relative to) another catheter or other tubular member, such as a coil, for example. This may lead to rubbing off of the visual markers from the catheter, and hence the markers may no longer function as positioning guides or warning indicators. There is a continued need for maintaining the markers on the catheter or any other medical device.

Embodiments of the disclosure are directed to devices and systems for maintaining visual markers on the catheter and preventing them from rubbing off. In some embodiments, the catheter may be indented in the locations where the visual markers are desired. The visual markers may be disposed within the indentations, thereby preventing the visual markers from contacting the endoscope, catheter, coil, or other tubular member or device used in conjunction with the catheter having visual markers.

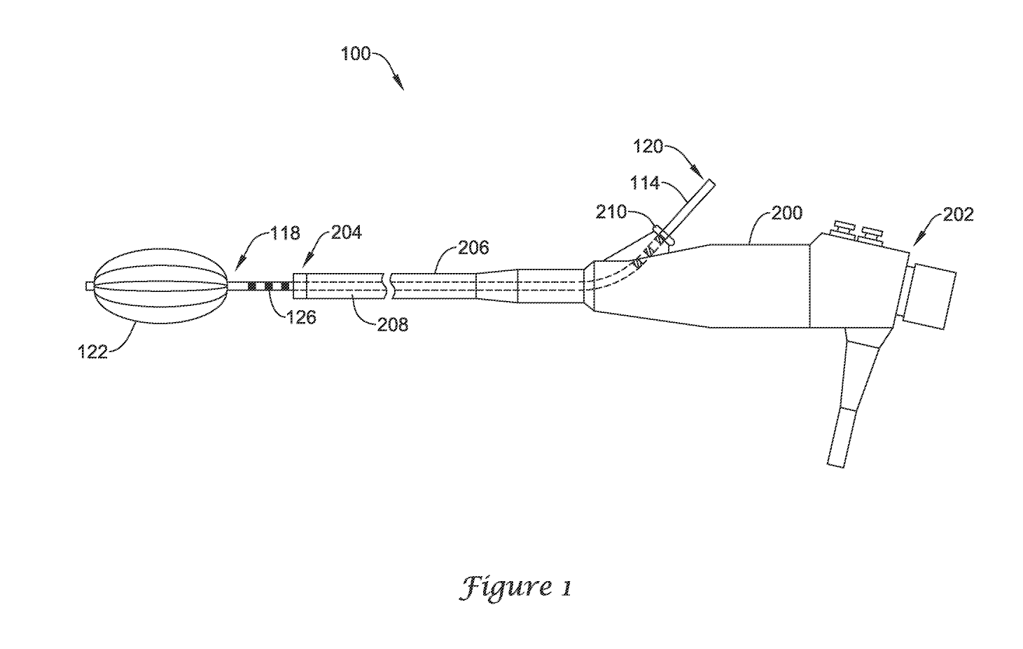

FIG. 1 illustrates an endoscopic system 100 including an endoscope 200, which may be formed in accordance with the description above. The endoscopic system 100 may include an elongated flexible tubular member 206 configured to examine a body cavity. The endoscope 200 includes a proximal end 202 and a distal end 204. A lumen 208 may extend through the tubular member 206, the lumen 208 connecting a proximal port 210 at or adjacent to the proximal end 202 and a distal port at the distal end 204. In some embodiments, the endoscope 200 may have more than one lumen 208 disposed therein and/or extending therethrough. The lumen 208 may be used to introduce one or more medical devices within a patient's body cavity. Endoscopes are well known in the art, and thus endoscope 200 is not discussed in further detail.

A catheter 114 may be inserted into the body cavity through the lumen 108 of endoscope 200 for diagnostic or therapeutic purposes. In some embodiments, the catheter 114 may be an elongated hollow, flexible tubular member having a lumen extending between a distal end 118 and a proximal end 120. The catheter 114 may be passed through the proximal port 210 to reach a target site. The catheter 114 may be configured to axially translate freely (move forward and backward) in the lumen 208 of the endoscope 200.

A therapeutic instrument 122 may be disposed at a distal end 118 of the catheter 114. In some embodiments, the therapeutic instrument 122 may include a basket having a number of electrodes arranged circumferentially on a plurality of struts. In some embodiments, the number of electrodes may be energized and used for ablation. For example, in some embodiments, the number of electrodes may be energized with radiofrequency energy to ablate tissue. Other therapeutic instruments are also contemplated.

The catheter 114 may include a number of visual markers 126 disposed on an outer surface of the catheter 114. In some embodiments, the visual markers 126 may be radiopaque to allow visualization of the catheter 114 using X-Ray imaging devices, or other imaging techniques. The visual markers 126 may be configured to be positioning guides and/or give visual indication of the position of the catheter 114 in the patient's body and/or relative to the endoscope 200, or relative to another catheter or tubular member.

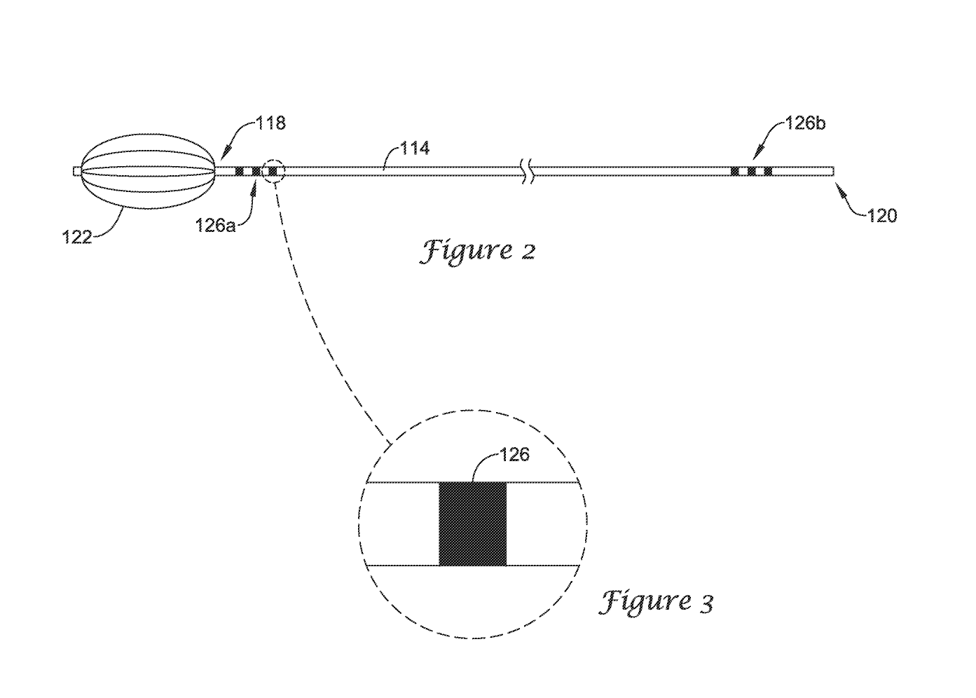

As shown in FIGS. 2 and 3, the visual markers 126 may be located proximate to the distal end 118 and the proximal end 120 of the catheter 114. For the purpose of illustration, the visual markers 126 may be further defined as a first series of visual markers 126A located adjacent the distal end 118 and a second series of visual markers 126B located adjacent the proximal end 120. In addition, the visual markers 126 may be circumferentially disposed about the catheter 114 so as to completely encircle the catheter 114. That is, the visual markers 126 may form 360 degree continuously circumferential visual markers 126 around the outer surface of the catheter 114. In some embodiments, the visual markers 126 may be discontinuously disposed about the circumference of the catheter 114.

In some embodiments, the visual markers 126 may be formed from ink, paint, colored pigment, or other suitable materials applied to the outer surface of the catheter 114. In some embodiments, one or more layers of one or more radiopaque materials may be applied to the outer surface of the catheter 114 to make the visual markers 126. Some examples of radiopaque materials that may be used include metals, metal flakes, metal powder, ceramics, ceramics powder, iodine, gold, platinum, palladium, tantalum, tungsten or tungsten alloy, barium sulfate (BaSO₄), bismuth subcarbonate (Bi₂O₂(CO₃)), bismuth trioxide (Bi₂O₃) or any other suitable radiopaque material, or a mixture of any of the above materials.

During operation, the catheter 114 may be translated multiple times into and out of the lumen 208 of the endoscope 200, or another catheter or tubular member. The visual markers 126 may frictionally bear or rub against the inner wall of the endoscope 200 (including the proximal port 210 and/or the distal end 204) and the layer of radiopaque material on the visual markers 126 may erode off the outer surface of the catheter 114 thereby removing the visual markers 126.

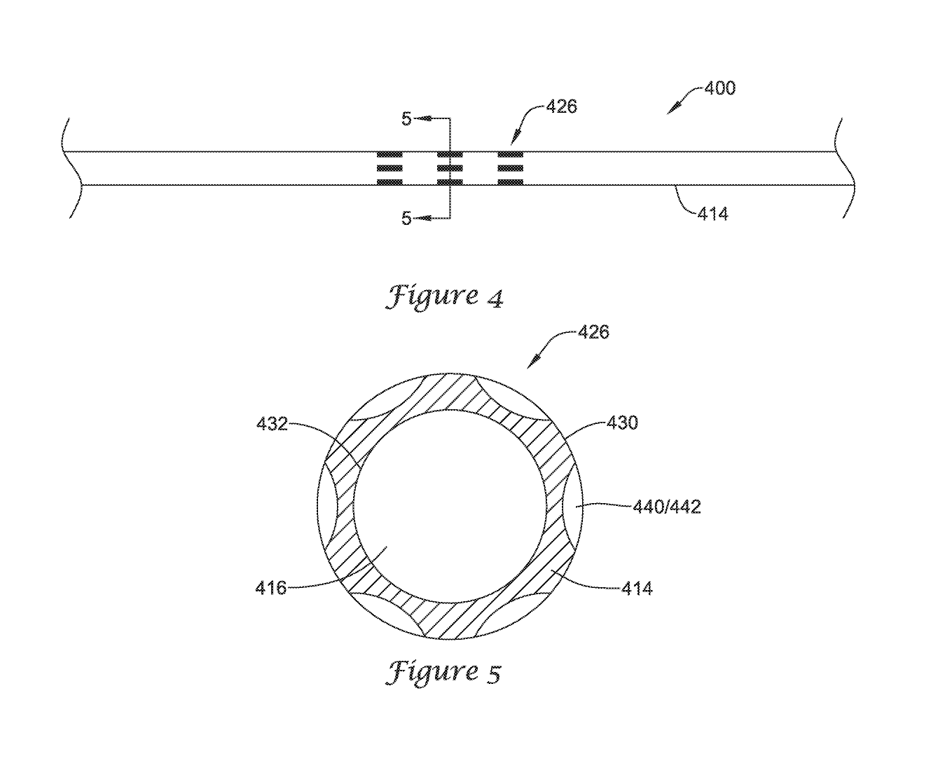

FIGS. 4 and 5 illustrate a portion of a catheter that may include features to prevent erosion of visual markers from an outer surface of the catheter. In some embodiments, a catheter 400 may include an elongate tubular member 414 having an outer wall 430 and an inner wall 432 defining a wall thickness, as seen for example in FIG. 5. Additionally, the inner wall 432 may define a lumen 416 extending longitudinally through the tubular member 414. The inner wall 432 and/or the lumen 416 may define an inner diameter of the tubular member 414. In some embodiments, the elongate tubular member 414 may be disposed over or about a catheter, a coil, or other tubular member and joined or fused thereto, such that the catheter, the coil, or the other tubular member defines the lumen and/or inner diameter thereof.

In some embodiments, the outer wall 430 of the tubular member 414 may include a first plurality of recessed portions 440 disposed in a distal section of the tubular member 414. Recessed portions 440 (and/or other recessed portions disclosed herein) may be formed using a suitable process such as crimping, mechanical cutting and/or scoring, etching, laser cutting and/or etching, stamping, or the like. In some embodiments, the first plurality of recessed portions 440 may be disposed about a circumference of the tubular member 414. In some embodiments, the first plurality of recessed portions 440 may be formed as a first series of circumferential bands in the outer wall 430. In some embodiments, the first series of circumferential bands and/or the first plurality of recessed portions 440 may be discontinuous about the circumference of the tubular member 414. In other words, the first plurality of recessed portions 440 may not each extend continuously about the circumference of the tubular member 414. In some embodiments, the first series of circumferential bands and/or the first plurality of recessed portions 440 may be longitudinally spaced apart along the tubular member 414 at one or more predetermined distances.

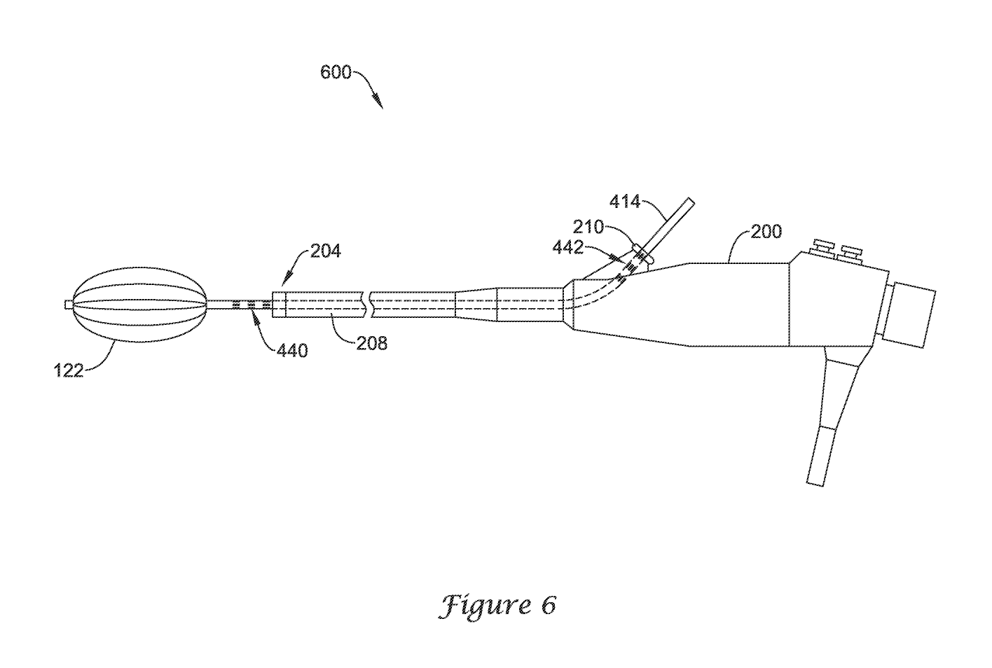

In some embodiments, the tubular member 414 may include a second plurality of recessed portions 442 disposed in a proximal section of the tubular member 414, similar to catheter 114 above, as may be seen in FIG. 6. In some embodiments, the second plurality of recessed portions 442 may be disposed about a circumference of the tubular member 414. In some embodiments, the second plurality of recessed portions 442 may be formed as a second series of circumferential bands in the outer wall 430. In some embodiments, the second series of circumferential bands and/or the second plurality of recessed portions 442 may be discontinuous about the circumference of the tubular member 414. In other words, the second plurality of recessed portions 442 may not each extend continuously about the circumference of the tubular member 414. In some embodiments, the second series of circumferential bands and/or the second plurality of recessed portions 442 may be longitudinally spaced apart along the tubular member 414 at one or more predetermined distances. In some embodiments, the one or more predetermined distances of the second series of circumferential bands and/or the second plurality of recessed portions 442 may be substantially equal to the one or more predetermined distances of the first series of circumferential bands and/or the first plurality of recessed portions 440. In other words, each one of the first series of circumferential bands (and/or first plurality of recessed portions 440) may correspond to one of the second series of circumferential bands (and/or second plurality of recessed portions 442), wherein the spacing between adjacent elements in the first series is the same as the spacing between corresponding adjacent elements in the second series.

As illustrated in FIG. 5, each of the first plurality of recessed portions 440 and similarly, each of the second plurality of recessed portions 442, may be formed as an indentation in the outer wall 430 of the tubular member 414. During use, non-indented surfaces of the outer wall 430 form a bearing surface that may contact and/or slide against an inner surface of the lumen 208 (including for example, the proximal port 210 and distal end 204) of the endoscope 200. In some embodiments, the first plurality of recessed portions 440 and/or the second plurality of recessed portions 442 may form a generally concave indentation below the outer wall 430 of the tubular member 414. Indentations of other suitable shapes are also contemplated. A visual marker 426 may be disposed within each of the first plurality of recessed portions 440 and/or each of the second plurality of recessed portions 442. For the purpose of clarity, each visual marker 426 disposed within one of the first plurality of recessed portions 440 may be defined as a first visual marker, and each visual marker disposed within one of the second plurality of recessed portions 442 may be defined as a second visual marker. The visual markers 426 may be formed using a variety of known methods including, but not limited to, crimping, pad printing, hot stamping, laser, adhesion, coating, painting, or other suitable methods. In some embodiments, each visual marker 426 may include a radiopaque component or element to facilitate visualization of the tubular member 414 using fluoroscopic imaging techniques to detect the location of the catheter 400 within the patient's body. Radiopaque visual markers may be advantageous in some embodiments where the tubular member 414 may be advanced deep into a body lumen and/or beyond the view of the endoscope.

In some embodiments, the first plurality of recessed portions 440 and/or the second plurality of recessed portions 442 may each include one, two, three, or more circumferential bands each consisting of a plurality of discrete, circumferentially spaced apart indentations disposed about the circumference of the tubular member 414. In some embodiments, the first plurality of recessed portions 440 and/or the second plurality of recessed portions 442 may extend only partially through the wall thickness of the tubular member 414. In other words, the first plurality of recessed portions 440 and/or the second plurality of recessed portions 442 may not extend completely through the wall thickness and/or the inner wall 432 of the tubular member 414. Additionally, in some embodiments, the circumferential bands of the first and second plurality of recessed portions may each include a marking with numbers, such as 5, 10, 15, or the like, indicating the relative depth of the tubular member 414 extending from the endoscope.

In some embodiments, a tubular member may include a third plurality of recessed portions, a fourth plurality of recessed portions, or other suitable quantities of recessed portions. In some embodiments, the recessed portions may be spaced along the length of the tubular member equally or at desired intervals. As discussed above with respect to the first plurality of recessed portions 440 and/or the second plurality of recessed portions 442, the third, fourth, or other plurality of recessed portions may each include a plurality of discrete, circumferentially spaced apart indentations disposed about the circumference of the tubular member. Other aspects and/or features consistent with the first and/or second plurality of recessed portions may also be provided. It is contemplated, however, that the third, fourth, or other plurality of recessed portions may include features different from those present in the first and/or second plurality of recessed portions while remaining consistent with and within the scope of the present disclosure.

FIG. 6 illustrates an endoscopic system 600 including an endoscope 200, as described above. The endoscope 200 may be configured to introduce and advance medical devices such as a catheter, biopsy forceps, tomes, and/or instruments for performing a medical procedure. The endoscope 200 may also include a light source and a camera objective disposed at the distal end 204 of the endoscope 200. The light source and the camera objective may enable the physician to observe internal organs of the patient's body lying distal of the distal end 204.

The endoscopic system may include an elongate tubular member 414 such as a catheter that may be advanced into the patient's body through the lumen 208 of the endoscope 200 to perform a medical procedure. In some embodiments, the endoscope 200 may be configured to slidably receive the elongate tubular member 414 within the lumen 208. The tubular member 414 may be configured to be extended and retracted in and out of the endoscope 200 through the lumen 208. The tubular member 414 may have a proximal section and a distal section. As discussed above, the tubular member 414 may include an outer wall 430 and an inner wall 432 defining a wall thickness.

In some embodiments, the distal section of the tubular member 414 may be configured to slide in and out of the distal end 204 of the endoscope 200 without the first visual markers disposed within the first plurality of recessed portions 440 contacting the distal end 204 of the endoscope 200. In some embodiments, each of the first plurality of recessed portions 440 is recessed from the outer wall 430 of the tubular member 414 a distance greater than a thickness of the first visual marker disposed therein. In some embodiments, the proximal section of the tubular member 414 may be configured to slide in and out of the proximal port 210 of the endoscope 200 without the second visual markers disposed within the second plurality of recessed portions 442 contacting the proximal port 210 of the endoscope 200. In some embodiments, each of the second plurality of recessed portions 442 is recessed from the outer wall 430 of the tubular member 414 a distance greater than a thickness of the second visual marker disposed therein. In other words, as the tubular member 414 slides in and out of the endoscope 200, the visual markers 426 are recessed from the outer wall 430 of the tubular member 414 and therefore spaced apart from inner surfaces of the endoscope 200 so as to protect the visual markers 426 from frictional contact with the endoscope 200 and prevent erosion or removal of the visual markers 426 from an outer surface of the tubular member 414.

Additionally, in some embodiments, the first plurality of recessed portions 440 and/or the second plurality of recessed portions 442 may provide tactile feedback to a user as the tubular member 414 is translated within the lumen 208 of the endoscope 200. As the first plurality of recessed portions 440 and/or the second plurality of recessed portions 442 are passed through the distal end 204 and/or the proximal port 210, respectively, the user may “feel” an interaction between edges of the recessed portions and the endoscope 200.

In some embodiments, the endoscope 200 may include a proximal port 210 in fluid communication with the lumen 208, such that when the elongate tubular member 414 passes through the proximal port 210, through the lumen 208, and extends distally from the distal end 204 of the endoscope 200, one or more of the first or second visual markers is disposed outside of the endoscope 200. In other words, at least one visual marker 426 may be visible outside of the endoscope 200 at all times. In some embodiments, a distance between a proximalmost first visual marker and a proximalmost second visual marker may be substantially equal to a distance between the proximal port 210 and the distal end 204 of the endoscope 200. In some embodiments, a distance between a distalmost first visual marker and a distalmost second visual marker may be substantially equal to a distance between the proximal port 210 and the distal end 204 of the endoscope 200. Other, intervening visual markers may be similarly positioned a distance apart that is substantially equal to a distance between the proximal port 210 and the distal end 204 of the endoscope 200.

Various embodiments disclosed herein are generally described in the context of intravascular devices. It is understood, however, that embodiments of the disclosure have applicability in other contexts, such as use within other vessels of the body, including other arteries, veins, and vasculature (e.g., cardiac and urinary vasculature and vessels), intraluminal spaces in the gastrointestinal tract or pulmonary system (i.e., esophagus, colon, duodenum, stomach, trachea, bronchus, etc.), and other tissues of the body, including various organs.

It is to be understood that even though numerous characteristics of various embodiments have been set forth in the foregoing description, together with details of the structure and function of various embodiments, this detailed description is illustrative only, and changes may be made in detail, especially in matters of structure and arrangements of parts illustrated by the various embodiments to the full extent indicated by the broad general meaning of the terms in which the appended claims are expressed.

What is claimed is:

1. A catheter, comprising:
an elongate tubular member having an outer wall and an inner wall defining a wall thickness, the inner wall further defining a lumen extending therethrough;
wherein the elongate tubular member includes a first plurality of recessed portions disposed in a distal section;
wherein the first plurality of recessed portions each includes a visual marker disposed therein.

2. The catheter of claim 1, wherein the first plurality of recessed portions is disposed about a circumference of the elongate tubular member.

3. The catheter of claim 2, wherein the first plurality of recessed portions is formed as a first series of circumferential bands in the outer wall.

4. The catheter of claim 3, wherein the first series of circumferential bands is discontinuous.

5. The catheter of claim 3, wherein the first series of circumferential bands is longitudinally spaced apart at one or more predetermined distances.

6. The catheter of claim 1, further including a second plurality of recessed portions disposed in a proximal section of the elongate tubular member.

7. The catheter of claim 6, wherein the second plurality of recessed portions is disposed about a circumference of the elongate tubular member.

8. The catheter of claim 7, wherein the second plurality of recessed portions is formed as a second series of circumferential bands in the outer wall.

9. The catheter of claim 8, wherein the second series of circumferential bands is discontinuous.

10. The catheter of claim 8, wherein the second series of circumferential bands is longitudinally spaced apart at one or more predetermined distances.

11. An endoscopic system, comprising:
an endoscope having a lumen extending therethrough, and having a light source and a camera objective each disposed at a distal end for observation distal of the distal end; and
an elongate tubular member having a first plurality of recessed portions disposed in a distal section thereof;
wherein each of the first plurality of recessed portions includes a first visual marker disposed therein;
wherein the endoscope is configured to slidably receive the elongate tubular member within the lumen.

12. The endoscopic system of claim 11, wherein the distal section of the elongate tubular member is configured to slide in and out of the distal end of the endoscope without the first visual markers contacting the distal end of the endoscope.

13. The endoscopic system of claim 11, wherein each of the first plurality of recessed portions is recessed from an outer wall of the elongate tubular member a distance greater than a thickness of the first visual marker disposed therein.

14. The endoscopic system of claim 11, wherein the first plurality of recessed portions is disposed about a circumference of the elongate tubular member.

15. The endoscopic system of claim 14, wherein the first plurality of recessed portions is formed as a first series of circumferential bands.

16. The endoscopic system of claim 15, wherein the first series of circumferential bands is discontinuous.

17. The endoscopic system of claim 15, wherein the first series of circumferential bands is longitudinally spaced apart at one or more predetermined distances.

18. The endoscopic system of claim 11, wherein the elongate tubular members included a second plurality of recessed portions disposed in a proximal section of the elongate tubular member, each of the second plurality of recessed portions having a second visual marker disposed therein.

19. The endoscopic system of claim 18, wherein the endoscope includes a proximal port in fluid communication with the lumen, such that when the elongate tubular member passes through the proximal port, through the lumen, and extends distally from the distal end of the endoscope, one or more of the first or second visual markers is disposed outside of the endoscope.

20. The endoscopic system of claim 19, wherein a distance between a proximalmost first visual marker and a proximalmost second visual marker is substantially equal to a distance between the proximal port and the distal end of the endoscope, or wherein a distance between a distalmost first visual marker and a distalmost second visual marker is substantially equal to the distance between the proximal port and the distal end of the endoscope.
